(19) United States
(12) Patent Application Publication
(10) Pub. No.: US 2026/0192091 A1
(43) Pub. Date: Jul. 9, 2026

(54) INTRAVENOUS LINE SLEEVE DEVICE

(71) Applicant: Gabriel Martinez, Mc Allen, TX (US)

(72) Inventor: Gabriel Martinez, Mc Allen, TX (US)

(21) Appl. No.: 19/015,243

(22) Filed: Jan. 9, 2025

(51) Int. Cl. A61M 25/02

(52) U.S. Cl. CPC A61M 25/02; A61M 2025/0206; A61M 2202/0007; A61M 2205/0216; A61M 2209/088; A61M 2210/08; A61M 2210/12

(57) ABSTRACT

An intravenous line sleeve device for securing an intravenous line on limb of a user includes an intravenous line that can be inserted into an artery of the limb of the user to deliver medication into the artery. A sleeve is continuously arcuate about a central axis of the sleeve such that the sleeve defines a cylindrical shape. The sleeve is comprised of an elastomeric material which can be stretched around the limb of the user. The sleeve is positionable over the intravenous line to inhibit the intravenous line from dangling on the limb of the user. The sleeve has an inner layer which is comprised of a fluid absorbent material to absorb perspiration on the limb of the user when the sleeve is worn around the limb of the user.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to sleeve devices and more particularly pertains to a new sleeve device for securing an intravenous line on a limb of a user. The device includes an intravenous line that is insertable into an artery of the limb of the user and a sleeve that is positionable around the limb of the user. The sleeve is comprised of an elastomeric material such that the sleeve can be stretched around the limb of the user and subsequently compress around the limb of the user to secure the intravenous line on the limb of the user. The sleeve includes an inner layer that is comprised of a fluid absorbent material to absorb perspiration from the user. The sleeve includes an outer layer that is comprised of a compressible material to enhance comfort for the user when the user grips the outer layer to position the sleeve around the limb of the user.

### (2) Description of Related Art Including Information Disclosed Under 37 CFR 1.97 and 1.98.

The prior art relates to sleeve devices including: a variety of arm cuff devices that each at least includes a cuff that can be wrapped around a limb of a user having the cuff being removably attached to itself to form a closed loop around the limb such that the cuff secures an intravenous line on the limb; an intravenous line securing device that includes a strap that has a first mating member and a second mating member which is matable to the first mating member such that the strap forms a closed loop around a limb of a user and a securing member attached to the second mating member which releasably engages an intravenous line; a intravenous needle protector device that includes a housing that is attachable to a limb of a user and an opening extending through the housing to accommodate an intravenous needle. In no instance does the prior art disclose an intravenous line sleeve device that includes an elastomeric sleeve which forms a cylinder which can be slid onto a limb of a user to secure an intravenous line on the limb and which has an inner layer comprised of a fluid absorbent material to absorb perspiration from the user.

### (h) BRIEF SUMMARY OF THE INVENTION

An embodiment of the disclosure meets the needs presented above by generally comprising an intravenous line that can be inserted into an artery of the limb of the user to deliver medication into the artery. A sleeve is continuously arcuate about a central axis of the sleeve such that the sleeve defines a cylindrical shape. The sleeve is comprised of an elastomeric material which can be stretched around the limb of the user. The sleeve is positionable over the intravenous line to inhibit the intravenous line from dangling on the limb of the user. The sleeve has an inner layer which is comprised of a fluid absorbent material to absorb perspiration on the limb of the user when the sleeve is worn around the limb of the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an intravenous line sleeve device according to an embodiment of the disclosure.

FIG. 2 is a top view of an embodiment of the disclosure.

FIG. 3 is a cross sectional view taken along line 3-3 of FIG. 1 of an embodiment of the disclosure.

FIG. 4 is a perspective in-use view of an embodiment of the disclosure.

### (j) DETAILED DESCRIPTION OF THE INVENTION

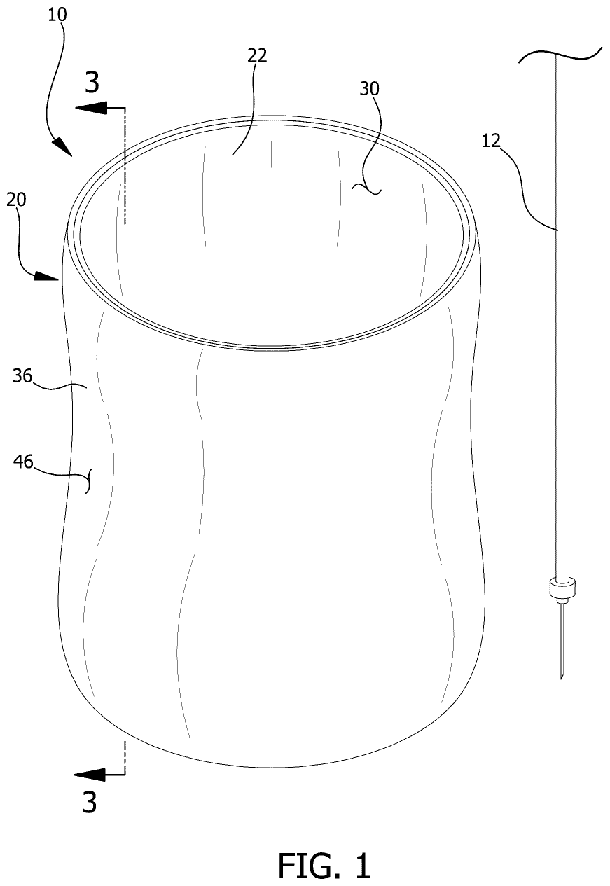

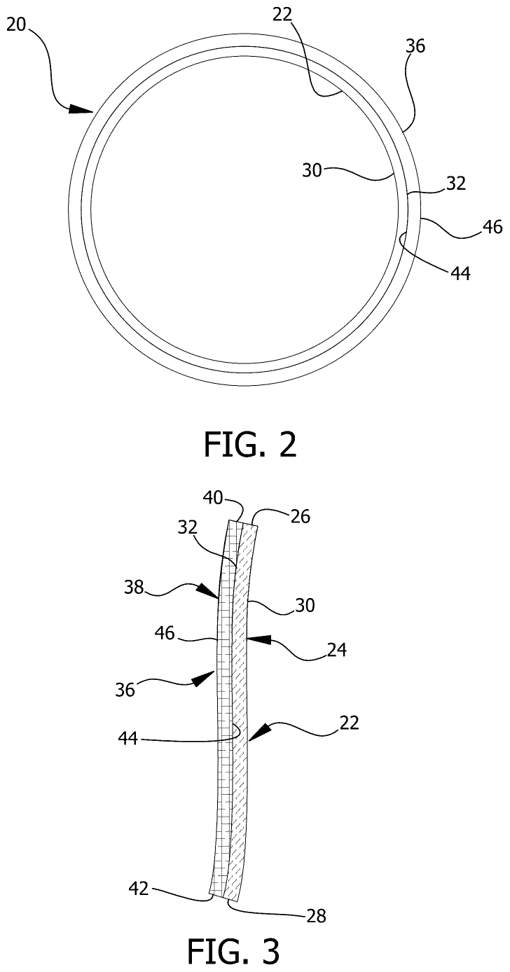

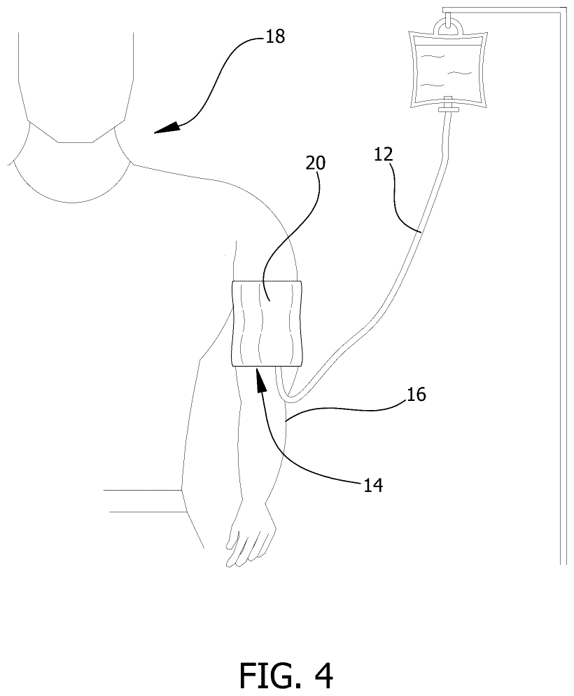

With reference now to the drawings, and in particular to FIGS. 1 through 4 thereof, a new sleeve device embodying the principles and concepts of an embodiment of the disclosure and generally designated by the reference numeral 10 will be described.

As best illustrated in FIGS. 1 through 4, the intravenous line sleeve device 10 generally comprises an intravenous line 12 which can be inserted into an artery 14 of the limb 16 of the user 18 to deliver medication into the artery 14. The user 18 may be an individual that is receiving medical care for an extended period of time, including but not being limited to an individual who is recovering from major surgery or an individual who is being treated for a terminal illness. Additionally, the intravenous line 12 may be a peripherally inserted central catheter or other type of intravenous line that is intended to remain in place for extended periods of time.

A sleeve 20 is provided that is continuously arcuate about a central axis of the sleeve 20 such that the sleeve 20 defines a cylindrical shape. The sleeve 20 is comprised of an elastomeric material such that the sleeve 20 can be stretched around the limb 16 of the user 18. Furthermore, the sleeve 20 is positionable over the intravenous line 12 such that the sleeve 20 inhibits the intravenous line 12 from dangling on the limb 16 of the user 18. The sleeve 20 has an inner layer 22 that is comprised of a fluid absorbent material such that said inner layer 22 absorbs perspiration on the limb 16 of the user 18.

The inner layer 22 has an outer wall 24 extending between a first end 26 and a second end 28. The outer wall 24 has an inwardly facing surface 30 and an outwardly facing surface 32; the inwardly facing surface 30 rests against skin 34 of the user 18 when the sleeve 20 is positioned around the limb 16 of the user 18. Furthermore, the fluid absorbent material of the inner layer 22 is comprised of the elastomeric material of the sleeve 12, including but not being limited to a blend of a synthetic textile material and spandex, such that the inner layer 22 can be stretched around the limb 16 of the user 18 and subsequently compress against the limb 16 of the user 18 to secure the intravenous line 12 on the limb 16 of the user 18. Furthermore, the fluid absorbent material of the inner layer 22 includes but is not being limited to a textile material, such as terry cloth or a wicking material, such that the inner layer 22 absorbs perspiration from the user 18 when the inner layer 22 is positioned around the limb 16 of the user 18.

The sleeve 20 comprises an outer layer 36 which has an exterior wall 38 extending between a primary end 40 and a secondary end 42 and the exterior wall 38 has an inwardly facing surface 44 and an outwardly facing surface 46. The inwardly facing surface 44 of the exterior wall 38 is bonded to the outwardly facing surface 32 of the outer wall 24 of the inner layer 22. Furthermore, the primary end 40 is aligned with the first end 26 and the secondary end 42 is aligned with the second end 28. The outer layer 36 is comprised of the elastomeric material of the sleeve 20, including but not being limited to a blend of a synthetic textile and spandex, such that the outer layer 36 can be stretched around the limb 16 of the user 18 in conjunction with the inner layer 22 and subsequently compress against the limb 16 of the user 18 in conjunction with the inner layer 22. Additionally, the elastomeric material of the outer layer 36 is comprised of a compressible material, including but not being limited to an organic textile such as cotton or wool, to enhance comfort for the user 18 when the user 18 grips the outer layer 36 to position the outer layer 36 around the limb 16 of the user 18.

In use, the intravenous line 12 is inserted into the artery 14 of the limb 16 of the user 18 with best practices employed by a medical professional and the sleeve 20 is positioned around the limb 16 of the user 18 such that the sleeve 20 is positioned over the intravenous line 12. Furthermore, the sleeve 20 compresses the intravenous line 12 against the skin 34 of the user 18 to inhibit the intravenous line 12 from dangling and potentially being dislodged or pulled as can commonly occur with unsecured intravenous line 12s. The inner layer 22 of the sleeve 20 absorbs perspiration from the user 18 to reduce the likelihood of chafing and also to inhibit the perspiration from collecting around the intravenous line 12 and potentially causing irritation or an infection. The sleeve 20 can be removed from the limb 16 of the user 18 at any time without disturbing the intravenous line 12 such that the intravenous line 12 can be serviced or removed or to enable various medication lines to be coupled to the intravenous line 12. Furthermore, the sleeve 20 can be laundered when the sleeve 20 becomes soiled with perspiration to enhance the cleanliness and comfort of the user 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An intravenous line sleeve device being positionable around a limb of a user, said device comprising:
an intravenous line being configured to be inserted into an artery of the limb of the user to deliver medication into the artery; and
a sleeve being continuously arcuate about a central axis of said sleeve such that said sleeve defines a cylindrical shape, said sleeve being comprised of an elastomeric material wherein said sleeve is configured to stretched around the limb of the user, said sleeve being positionable over said intravenous line wherein said sleeve is configured to inhibit said intravenous line from dangling on the limb of the user, said sleeve having an inner layer being comprised of a fluid absorbent material wherein said inner layer is configured to absorb perspiration on the limb of the user when said sleeve is worn around the limb of the user.

2. The intravenous line sleeve device according to claim 1, wherein:
said inner layer has an outer wall extending between a first end and a second end; and
said outer wall has an inwardly facing surface and an outwardly facing surface wherein said inwardly facing surface is configured to rest against skin of the user when said sleeve is positioned around the limb of the user.

3. The intravenous line sleeve device according to claim 2, wherein said fluid absorbent material of said inner layer is comprised of said elastomeric material of said sleeve wherein said inner layer is configured to be stretched around the limb of the user and subsequently compress against the limb of the user to secure said intravenous line on the limb of the user.

4. The intravenous line sleeve device according to claim 2, wherein:
said sleeve comprises an outer layer having an exterior wall extending between a primary end and a secondary end;
said exterior wall has an inwardly facing surface and an outwardly facing surface;
said inwardly facing surface of said exterior wall is bonded to said outwardly facing surface of said outer wall of said inner layer having said primary end being aligned with said first end and having said secondary end being aligned with said second end.

5. The intravenous line sleeve device according to claim 4, wherein said outer layer is comprised of said elastomeric material of said sleeve wherein said outer layer is configured to be stretched around the limb of the user in conjunction with said inner layer and subsequently compress against the limb of the user in conjunction with said inner layer.

6. The intravenous line sleeve device according to claim 5, wherein said elastomeric material of said outer layer is comprised of a compressible material wherein said outer layer is configured to enhance comfort for the user when the user grips said outer layer to position said outer layer around the limb of the user.

7. An intravenous line sleeve device being positionable around a limb of a user, said device comprising:
an intravenous line being configured to be inserted into an artery of the limb of the user to deliver medication into the artery; and
a sleeve being continuously arcuate about a central axis of said sleeve such that said sleeve defines a cylindrical shape, said sleeve being comprised of an elastomeric material wherein said sleeve is configured to stretched around the limb of the user, said sleeve being positionable over said intravenous line wherein said sleeve is configured to inhibit said intravenous line from dangling on the limb of the user, said sleeve having an inner layer being comprised of a fluid absorbent material wherein said inner layer is configured to absorb perspiration on the limb of the user when said sleeve is worn around the limb of the user, said inner layer having a outer wall extending between a first end and a second end, said outer wall having an inwardly facing surface and an outwardly facing surface wherein said inwardly facing surface is configured to rest against skin of the user when said sleeve is positioned around the limb of the user, said fluid absorbent material of said inner layer being comprised of said elastomeric material of said sleeve wherein said inner layer is configured to be stretched around the limb of the user and subsequently compress against the limb of the user to secure said intravenous line on the limb of the user, said sleeve comprising an outer layer having an exterior wall extending between a primary end and a secondary end, said exterior wall having an inwardly facing surface and an outwardly facing surface, said inwardly facing surface of said exterior wall being bonded to said outwardly facing surface of said outer wall of said inner layer having said primary end being aligned with said first end and having said secondary end being aligned with said second end, said outer layer being comprised of said elastomeric material of said sleeve wherein said outer layer is configured to be stretched around the limb of the user in conjunction with said inner layer and subsequently compress against the limb of the user in conjunction with said inner layer, said elastomeric material of said outer layer being comprised of a compressible material wherein said outer layer is configured to enhance comfort for the user when the user grips said outer layer to position said outer layer around the limb of the user.

8. A method of securing an intravenous line to a limb of a user, the steps of the method comprising:
inserting an intravenous line into an artery of the limb of the user;
stretching a sleeve around the limb of the user, said sleeve being comprised of an elastomeric material wherein said sleeve is configured to compress against the limb of the user subsequent to said sleeve being stretched around the limb of the user; and
positioning said sleeve over said intravenous line such that an inner layer of said sleeve compresses against said intravenous line wherein said sleeve is configured to secure the intravenous line to the limb of the user, said inner layer being comprised of a fluid absorbent material wherein said inner layer is configured to absorb perspiration from the user.
